(19) United States
(12) Patent Application Publication
(10) Pub. No.: US 2008/0286238 A1
(43) Pub. Date: Nov. 20, 2008

(54) Recombinant Adenovirus Expressing A Gene Encoding Streptolysin O Proetin and Anti-Cancer Composition Comprising Same

(75) Inventors: Chul Woo Kim, Seoul (KR); Wan Seok Yang, Seoul (KR); Min Kyoung Kim, Seoul (KR)

Correspondence Address: SUGHRUE MION, PLLC, 2100 PENNSYLVANIA AVENUE, N.W., SUITE 800, WASHINGTON, DC, 20037, US

(73) Assignees: Chul Woo Kim, Seoul (KR); Bioinfra Inc., Yeongeon-dong, Jongro-gu, Seoul (KR)

(21) Appl. No.: 12/091,504

(22) PCT Filed: Oct. 26, 2006
(86) PCT No.: PCT/KR2006/004394

§ 371 Date: Apr. 25, 2008

(30) Foreign Application Priority Data
Oct. 26, 2005 (KR) 10-2005-0101091

(51) Int. Cl. A61K 35/76; C12N 7/00; A61P 31/00

(52) U.S. Cl. 424/93.2; 435/235.1

(57) ABSTRACT

A recombinant adenovirus expressing a streptolysin O (SLO) protein comprising a SLO gene; a promoter operably linked to the SLO gene; a polyadenylation signal sequence; and an adenovirus genome lacking E1 gene effectively kills a cancer cell by expressing a pore-forming toxin, SLO protein, and, therefore, is useful for the suicide cancer gene therapy.

### FIELD OF THE INVENTION

The present invention relates to a recombinant adenovirus expressing a gene encoding a streptolysin O (SLO) protein and an anti-cancer composition comprising same as an active ingredient.

### BACKGROUND OF THE INVENTION

Suicide gene therapy has received much attention in cancer biology as an alternative therapy to conventional chemotherapy and radiotherapy (see [Gottesman M M, Cancer Gene Ther., 10:501-8, 2003]). Typically, suicide cancer gene therapy involves specific delivery of various cytotoxic genes, such as apoptotic factors or enzyme-prodrug combinations, to cancer cells. The subsequent expression of these genes then induces cell death. Suicide cancer gene therapies based on apoptotic factors such as p53 (see [Vecil G G and Lang F F, J. Neurooncol., 65:237-46, 2003]), FasL (see [Sudarshan S, et al., Cancer Gene Ther., 12:12-8, 2005]), Bax (see [Ozawa T, et al., Cancer Gene Ther., 12:449-55, 2005]), and tumor necrosis factor-related apoptosis-inducing ligand (TRAIL) (see [Shi J, et al., Cancer Res., 65:1687-92, 2005]) have been extensively studied in athymic mice models. However, they have intrinsic limitations as anti-cancer gene therapeutic reagents because cancer cells evolve to gain resistance to such apoptotic insults. For example, anti-apoptotic molecules like Bcl-2, Bcl-X_{L}, c-FILP, c-IAP, and survivins are often overexpressed in many types of cancer cells, and these confer on cancer cells resistance against apoptotic factors (see [Igney F H and Krammer P H, Nat. Rev. Cancer, 2:277-88, 2002]).

Enzyme-prodrug systems for suicide gene therapy are represented by the herpes simplex virus thymidine kinase/ganciclovir (HSV-tk/GCV) combination, the cytosine deaminase/5-fluoro-cytosine combination, and the cytochrome P450/cyclophosphamide combination systems. These kill the targeted cancer cells by interfering with the DNA/RNA synthesis processes. The toxic substances produced by these combinations can spread out to the neighboring cancer cells and induce consecutive cell death (the bystander effect). Two drawbacks of these enzyme-prodrug systems are: the prominent bystander effect could cause unwanted toxic effect to neighboring normal cells; and these systems are less effective against cancer cells that are not actively dividing. Therefore, the present inventors sought to develop a novel suicide gene therapeutic reagent that can overcome the anti-apoptotic resistance and has anti-cancer activity which does not dependent on the cell proliferation rate.

Streptolysin O (SLO) is a toxin secreted by bacteria from the genus Streptococcus and is a prototype member of pore-forming bacterial cytolysins along with Staphlyococcal α-toxin and Escherichia coli hemolysin (see [Bhakdi S, et al., Arch. Microbiol., 165:73-9, 1996]). SLO is a single polypeptide chain having a molecular weight of about 62 kDa. It binds specifically to membrane cholesterol, and oligomerizes to create a 45- to 50-membered ring structure, which penetrates into the membrane to make a large pore diameter of 25 to 30 nm.

Cell biologists have exploited the pore-forming property of SLO for macromolecule delivery, as evidenced by a large number of published applications (see [Garcia-Chaumont C, et al., Pharmacol. Ther., 87:255-77, 2000; Tarassishin L, et al., Proc, Natl. Acad. Sci. U.S.A., 101:17050-5, 2004; and Walev I, et al., Proc. Natl. Acad. Sci. U.S.A., 98:3185-90, 2001]). When the cell membrane is treated with SLO, large membrane pores are generated and the membrane becomes permeable to extracellular DNA, RNA, peptides, and proteins. Thus, the presence of SLO-induced pores in a cell membrane will result in cytolysis because of the loss of the balance between influxes and effluxes across the cell membrane. In addition, it has been reported that SLO increases the bacterial virulence by inactivating macrophages and phagocytes (see [Sierig G, et al., Infect. Immun. 71:446-55, 2003]).

The pore-forming toxin such as magainin, cecropin and verotoxin has been used as an anti-cancer agent in the form of immune toxin or natural protein (see [Farkas-Himsley H, et al., Proc. Natl. Acad. Sci. U.S.A. 92:6996-7000, 1995; Moore A J, et al., Pept. Res. 7:265-9, 1994; and Ohsaki Y, et al., Cancer Res. 52:3534-8, 1992]), but it has not been reported that SLO can be used as an anti-cancer agent.

The present inventors sought to develop a novel powerful suicide gene therapeutic reagent that can overcome the anti-apoptotic resistance of cancer cells and maintains its activity regardless of the cellular proliferation rate.

### SUMMARY OF THE INVENTION

Accordingly, it is an object of the present invention to provide a recombinant adenovirus expressing a gene encoding a SLO protein and an anti-cancer composition comprising same as an active ingredient, which is capable of overcoming the anti-apoptotic resistance and maintains its apoptotic activity regardless of the cell proliferation rate.

In accordance with one aspect of the present invention, there is provided a recombinant adenovirus expressing a gene encoding a SLO protein in animal cells.

In accordance with another aspect of the present invention, there is provided an anti-cancer composition comprising said recombinant adenovirus as an active ingredient.

### BRIEF DESCRIPTION OF DRAWINGS

The above and other objects and features of the present invention will become apparent from the following description of the invention taken in conjunction with the following accompanying drawings, which respectively show:

FIG. 1A: Morphological change of 293T cells transfected with a SLO recombinant vector in a transient transfection system of the SLO gene;

FIG. 1B: Green fluorescence intensity of 293T cells co-transfected with a GFP reporter plasmid and a SLO-expressing recombinant vector measured by FACS;

FIG. 1C: Expression of the SLO in 293T cells transfected with the SLO recombinant vector measured by western blot analysis;

FIG. 2A: Amount of released cytosolic LDH into culture medium from 293T cells transfected with the SLO recombinant vector;

FIG. 2B: Amount of PI dye uptaken by 293T cells transfected with the SLO recombinant vector measured by FACS;

FIG. 2C: Morphological change of 293T cells transfected with the SLO recombinant vector;

FIG. 3A: Activity of cellular caspase-3 in 293T cells transfected with a the recombinant vector;

FIG. 3B: Expression of the SLO in 293T cells transfected with the SLO recombinant vector measured by western blot analysis;

FIG. 3C: Inhibition of cell death by cellular anti-apoptotic protein in 293T cells transfected with the SLO recombinant vector measured by FACS;

FIG. 4A: Diagrams of SLO protein deletion mutants of the present invention;

FIG. 4B: Expression of each deletion mutant in 293T cells transfected with each SLO protein deletion mutant measured by western blot analysis;

FIG. 4C: Amount of released cytosolic LDH into culture medium from 293T cells transfected with each SLO protein deletion mutant;

FIG. 4D: Percentage of cells having sub-genomic DNA contents in 293T cells transfected with each SLO protein deletion mutant;

FIG. 5A: Diagram of a shuttle vector to make a SLO recombinant adenovirus of the present invention;

FIG. 5B: Cre-inducible expression of GFP in C33A cells transfected with a recombinant adenovirus Ad-loxP-GFP with or without AdCreM2;

FIG. 5C: Cell death induced by Cre-inducible expression of the SLO in C33A cells transfected with a recombinant adenovirus Ad-loxP-GFP with or without AdCreM2;

FIG. 6: Extracellular anti-cancer activity of the SLO-expressing recombinant adenovirus of the present invention in various cancer cell lines; and

FIG. 7: Intracellular anti-cancer activity of the SLO-expressing recombinant adenovirus of the present invention in a human tumor derived from a human tumor xenograft.

### DETAILED DESCRIPTION OF THE INVENTION

In the present invention, “cell death” represents the phenomenon of cells dying through either of two distinct processes, “necrosis” or “apoptosis”. The necrosis means the death of living cells or tissues due to unexpected and accidental cell damage. Necrotic cells swell by influx of water, holes appear in the plasma membrane and intracellular materials spill out into the surrounding environment. Whereas, the apoptosis represents the cell death controlled by genes, which is essential for the developmental process and maintenance of cellular homeostasis.

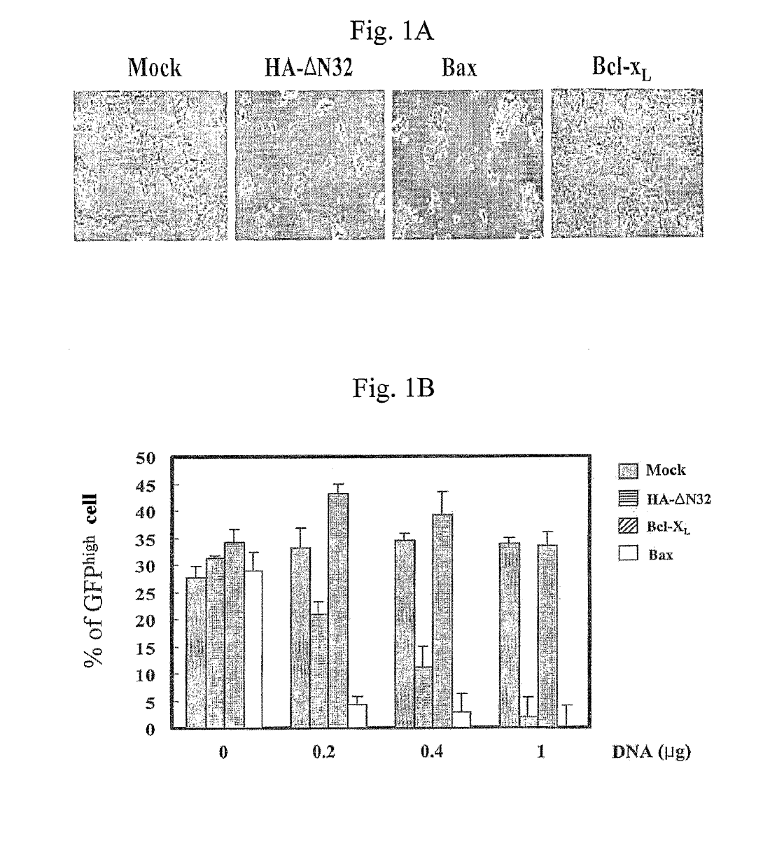

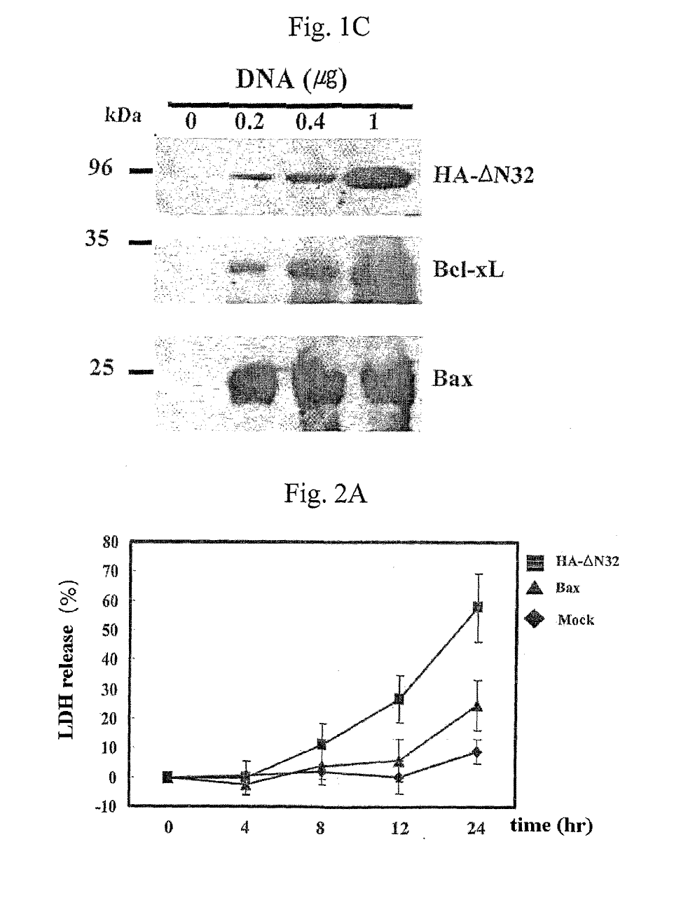

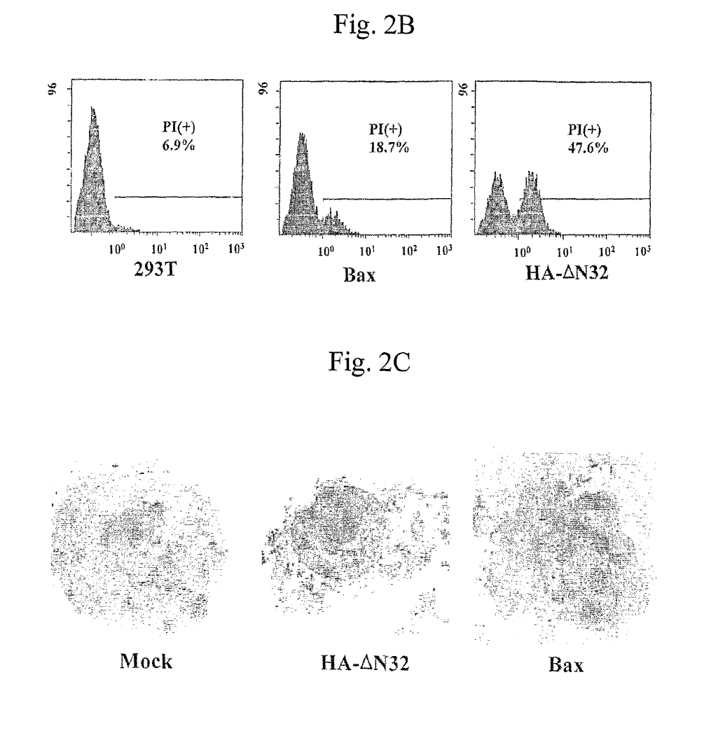

The present inventors have initially found that a SLO expressed in mammalian cells, which is typically expressed from the genus Streptococcus, possess the same cytotoxicity as a native bacterial SLO toxin (see FIGS. 1A to 1C), and the cytotoxicity is the result of an increase in the membrane permeability caused by plasma membrane pores generated by the SLO expressed in mammalian cells (see FIGS. 2A and 2B). In contrast to the cells transfected with the proapoptotic protein Bax, the membrane integrity of SLO-transfected cells rapidly disintegrates, which implies that the cell death caused by the SLO protein is activated by a cell death pathway other than apoptosis.

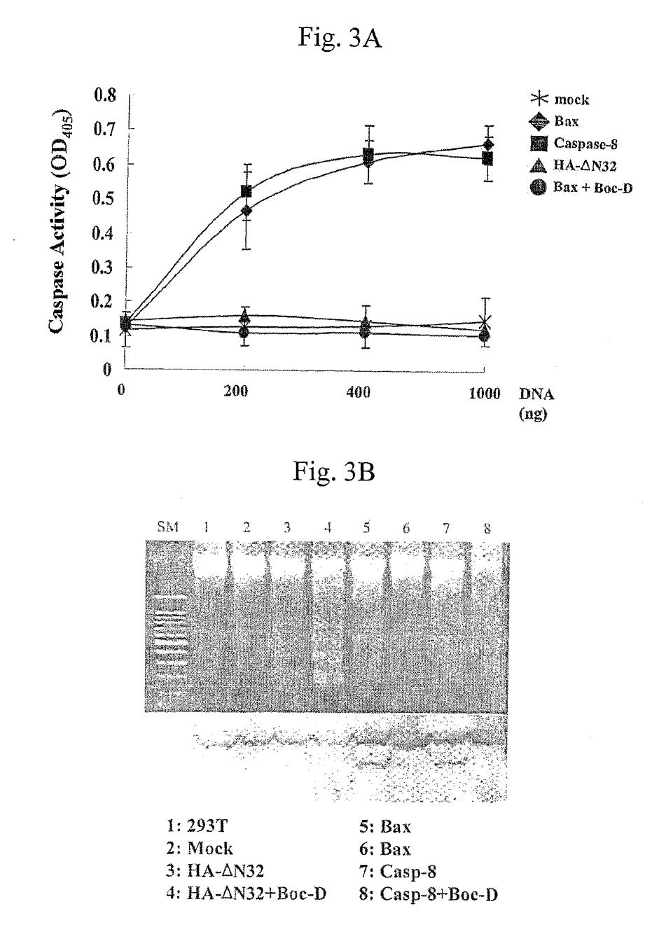

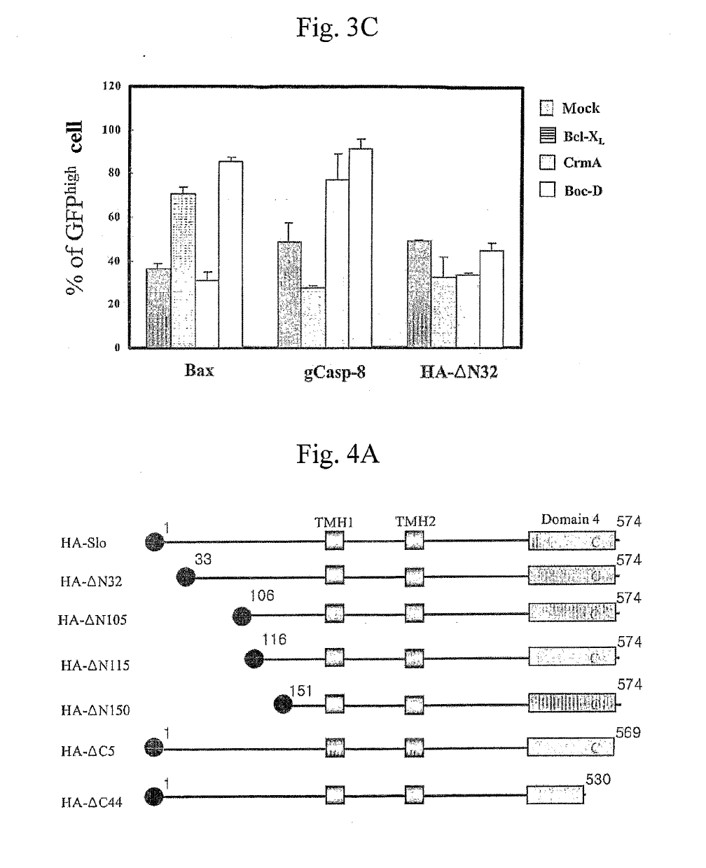

The ultrastructure of SLO-transfected cells monitored under the electron microscope shows many vacuoles and a less dense cytoplasm. This implies that the SLO induces nonapoptotic cell death, necrosis, by creating pore in the plasma membrane and increasing the membrane permeability (see FIG. 2C). The SLO-induced cell death is not dependent on caspase (see FIG. 3A) and is not affected by the overexpression of potent anti-apoptotic molecules (see FIGS. 3A to 3C). As a result, the plasma membranes of SLO-expressing mammalian cells are rendered permeable, and cytoplasmic contents leak out of cells, leading to plasma membrane disintegration. These findings coincide with the characteristics of cell death induced by a native SLO toxin and suggest that the SLO protein synthesized within mammalian cells acts in a way similar to a native SLO toxin.

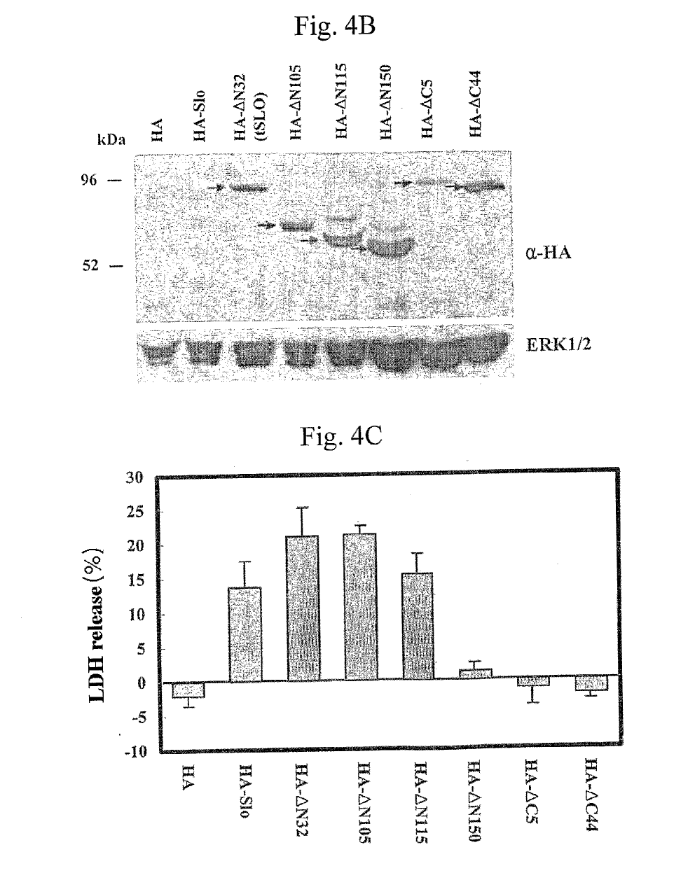

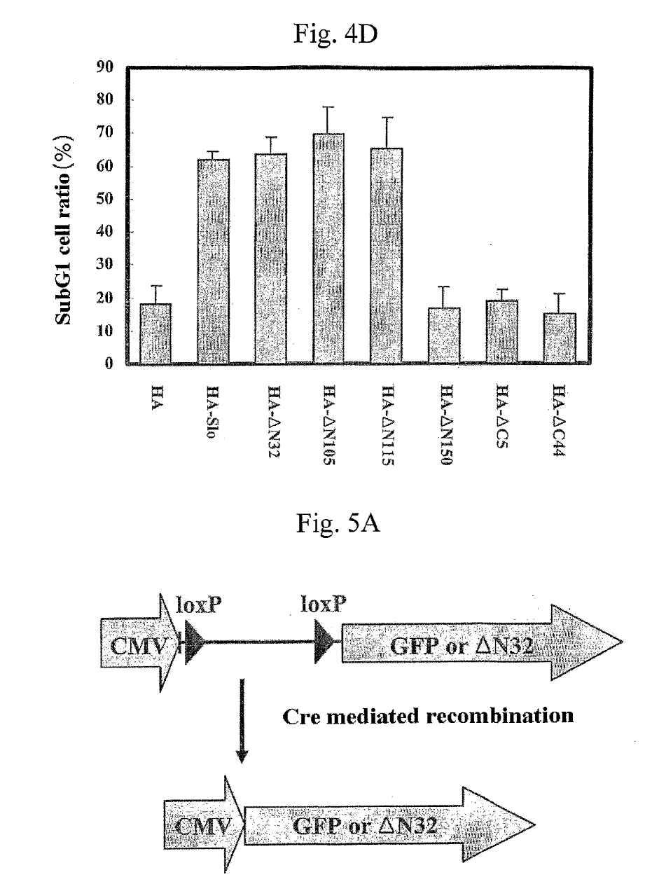

Further, several deletion mutants of the SLO were generated in order to identify the region responsible for the observed cell death, and the deletion of only 5 amino acids from the C-terminal region of the SLO leads to the lose of its cell-killing activity, whereas the N-terminal region up to 115 amino acids can be deleted without changing its cell-killing activity (see FIG. 4A).

Specifically, when the SLO deletion mutants are expressed in a mammalian cell, the deletion of up to 115 amino acids from the N-terminus induced cell death, but the deletion of up to 150 amino acids from the N-terminus negated the cell-killing activity of the SLO almost completely. Thus, the critical region for the SLO-induced membrane permeabilization is located between the 116^{th} and 150^{th} amino acids from N-terminus of the SLO protein (see FIGS. 4C and 4D). However, in contrast to the N-terminus, the deletions of only 5 amino acids from the C-terminus negated the cell-killing activity of the SLO (see FIGS. 4C and 4D).

In order to use the SLO protein confirmed to have a potent cell-killing activity in a mammalian cell as a suicide gene therapeutic reagent for tumors, the present invention provides a recombinant adenovirus, which can express the SLO gene in a mammalian cell.

The recombinant adenovirus of the present invention is characterized in that it comprises the SLO gene; a promoter connected to said gene; polyadenylation signal sequence; and an adenovirus genome lacking E1 gene.

The SLO gene used in the present invention may be a previously reported native sequence or a modified nucleotide sequence thereof having the same function, e.g., the reported SLO sequence (GenBank accession number: AB0505250) derived from Streptococcus pyogene (ATCC 700294D). Further, as confirmed in the deletion assay, the SLO gene is preferred to comprise a polynucleotide encoding the 116^{th} to 574^{th} amino acids of the amino acid sequence of the SLO protein (Genbank accession No. AB0505250). Although the full length SLO protein cause cell death of the transfected cells by permeabilizing the cellular membrane, the N-terminal region thereof is cut out by post-translational step. Thus, used in the present invention is a truncated version of a SLO having an amino acid sequence of SEQ ID NO: 3 which lacks the 32 N-terminal amino acids of the full-length SLO (574 amino acids). The truncated SLO is represented by the nucleotide sequence of SEQ ID No: 4.

The recombinant adenovirus may be prepared by inducing in vivo homologous recombination by co-transfecting a mammalian cell with a recombinant expression vector comprising the SLO gene and a viral vector comprising an adenovirus genome lacking E1.

First, in order to generate a recombinant expression vector comprising a SLO-expressing cassette, the SLO gene or a fragment thereof may be cloned into a shuttle vector such as a viral vector such as an adenoviral vector, adeno-associated viral vector and retroviral vector, and other conventional non-viral vectors.

The recombinant adenovirus of the present invention is prepared by inserting the SLO-expressing cassette of the recombinant expression vector into the viral genome by homologous recombination after co-transfecting a package cell line with the recombinant expression vector and a parental viral vector. For example, vmdl324Bst, pBHG10 or pJM17E1a can be used as a parental vector, and a 293T cell line can be used as a package cell line, but it is not intended to limit the scope of the invention. The parental vector which can not proliferate by itself due to the absence of E1 gene can proliferate the transfected virus by the package cell comprising the E1 gene.

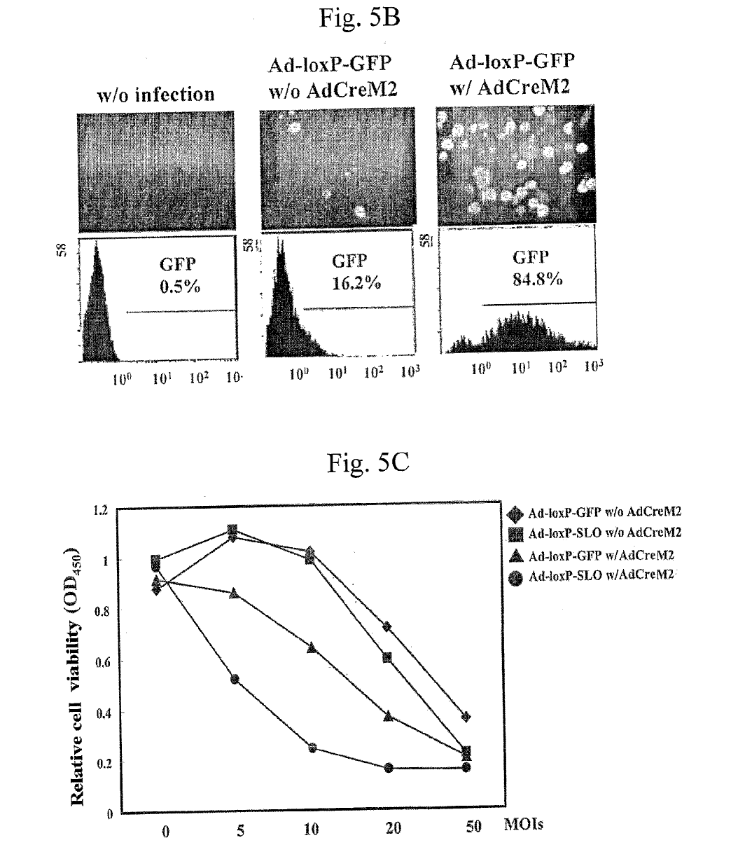

In one embodiment of the present invention, a replication-deficient adenovirus that can express a fragment of the SLO is prepared. The cDNA encoding a truncated version of the SLO lacking the 32 N-terminal amino acids of the full-length SLO is cloned into shuttle vector pCA14-loxP which contains an expression cassette comprising an immediately early promoter of cytomegalovirus, a multi-cloning site and late polyadenylation of SV40 to generate pCA14-loxP-SLO construct (see FIG. 5A). The recombinant expression vector pCA14-loxP-SLO is co-transfected with an adenoviral vector vmdl324Bst containing the Ad5 genome lacking E1 and E3 regions into E. coli to induce homologous recombination. The intended homologous recombinant adenoviral plasmid DNA is verified and transfected into 293T cells for virus packaging to generate a SLO-expressing recombinant adenovirus.

Generally, the transduction of an adenovirus genome encoding the SLO into a host cell causes host cell death before the adenovirus formation, and thus, the adenovirus formation does not take place. Because the SLO protein is a cytotoxic protein, in the present invention, a Cre-inducible expression system that is widely used for toxic gene expression to circumvent the difficulty of virus packaging of the SLO in a host cell is used to produce an adenovirus in a host cell having no Cre enzyme such that the adenovirus genome is modified not to express the SLO gene without a Cre enzyme. The Cre enzyme is an enzyme causing recombination, which recognizes a specific base pair of DNA (called “loxP”) and the DNA fragment presented containing in the base pair is deleted and the remaining portions are re-ligated (see FIG. 5A). Accordingly, a cassette in the form e.g., ‘loxP-nucleotide sequence not expressing protein-loxp’ is inserted between the promoter and the SLO gene to make the adenovirus genome not to express the toxic SLO gene, and the ‘loxP-nucleotide sequence not expressing protein-loxp’ cassette is deleted by recombination when the Cre enzyme is introduced to express the toxic SLO by a promoter.

The co-transfection of prepared a recombinant adenovirus Ad-loxP-SLO with Cre-expressing adenovirus results in marked cancer cell death, while the transfection of Ad-loxP-SLO without Cre-expressing adenovirus showed little cytotoxicity (see FIGS. 5B and 5C). Therefore, the recombinant adenovirus of the present invention expresses the SLO protein under the control of the Cre-inducible expression system.

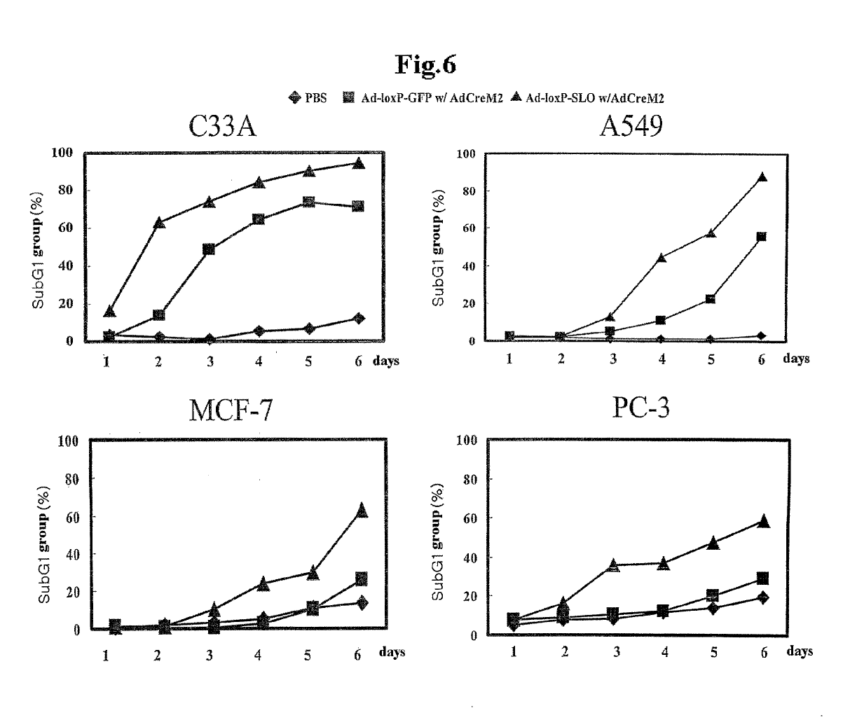

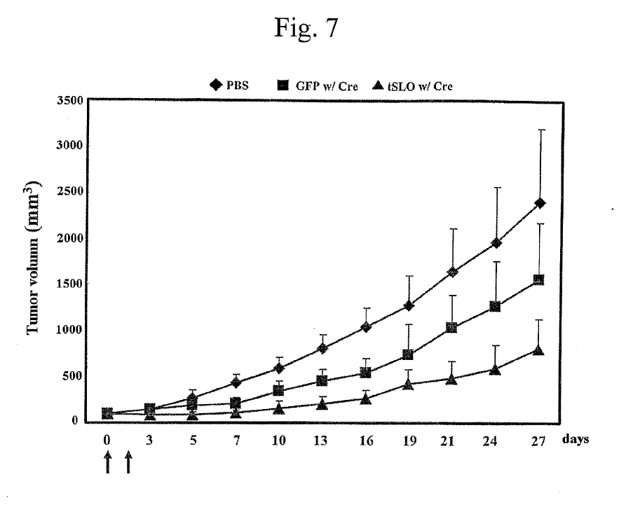

Further, the SLO-expressing recombinant adenovirus of the present invention shows an excellent cell-killing activity in all cancer cell lines examined (see FIG. 6), and the tumor growth is significantly inhibited by co-injection of Ad-loxP-SLO and Cre-expressing adenovirus into a human cervical cancer cell xenograft-derived tumors grown in a nude mouse model (see FIG. 7).

Accordingly, the present invention provides a pharmaceutical anti-cancer composition comprising the SLO-expressing recombinant adenovirus showing the excellent cell-killing activity as an active ingredient.

The composition may further comprise one or more pharmaceutically acceptable carriers. An excipient or diluent as the carrier may be selected from the group consisting of a saline solution, buffered saline solution, dextrose, water, glycerol and ethanol, but it is not intended to limit the scope of the invention. The composition may be administered parenterally, preferably by injection, for example, by intratumoral injection into cancer cells. The composition may be administered in an amount used in a conventional cancer gene therapy, preferably, ranging from about maximum of 1×10¹² to 1×10¹³ particles of a recombinant adenovirus, but it is not intended to limit the scope of the invention.

However, the foregoing dosage may be determined by preclinical or clinical trial, and change in consideration of idiosyncrasy and weight of the patient, kind and seriousness of illnesses, characteristics of the drug and interval and duration of drug.

Further, the SLO-expressing recombinant adenovirus or the composition comprising thereof can be injected into cancer cells to kill the cells by expressing the SLO protein.

The SLO-expressing recombinant adenovirus of the present invention is a replication deficient virus that can overexpress or secret the SLO protein inside of the cell when delivered to cancer cells. Then the overexpressed SLO protein increase the plasma membrane permeability by pore-forming, and cytoplasmic contents leaks out of cells, which eventually die due to plasma membrane disintegration. The SLO-induced cell death of the present invention damages the plasma membrane directly, and thus, the anti-apoptotic machinery developed during cancer cell evolution is unable to inhibit the cell death. Further, as SLO-induced cell death is not dependent on the cellular proliferation rate, cancer gene therapy based on the SLO gene may be effective against tumors with low proliferation rates, such as prostate cancer.

The following Examples are intended to further illustrate the present invention without limiting its scope.

### EXAMPLE 1

### Assay of Cell-Killing Activity of SLO

### <1-1> Construction of SLO Transient Expression Plasmid

In order to examine whether a SLO expressed in a mammalian cell can induce cell death by showing the cytolytic activity identical to that of an active, natural SLO toxin, a transient expression plasmid system of the SLO was constructed.

To clone the SLO gene from a genomic DNA of Streptococcus pyogenes (ATCC 700294D), PCR was performed using ExTaq Polymerase (Takara bio, Japan) and a pair of primers (SEQ ID NOs: 1 and 2). The PCR condition was 30 cycles of 5 min at 94° C., 1 min at 58° C. and 1 min at 72° C. after initial denaturation for 10 min at 94° C., and final extension for 1 min at 72° C. The amplified DNA fragment was subcloned into EcoRI/XhoI restriction site of vector pcDNA3 (Invitrogen) to obtain vector pcDNA3-SLO, and the subcloned SLO fragment was analyzed by the dideoxynucleotide sequencing method.

As can be seen in Table 1, the sequence analysis revealed that the SLO gene cloned in vector pcDNA3 differs by 20 nucleotides from the reported SLO sequence (Genbank accession number, AB050250), but the amino acids encoded therein were identical to those of the reported SLO sequence.

TABLE 1

| Nucleotide | | Amino acid | |
|---|---|---|---|
| mutation | | mutation | |
| (#AB050250→ | Position of mutated | (#AB050250→ | Position of |
| cloned SLO gene) | nucleotide | cloned SLO gene) | amino acid |
| cta→ctg | 60 | | |
| aat→aac | 84 | | |
| agt→aat | 149 | S→S | 50 |
| atc→act | 185, 186 | I→I | 62 |
| atg→acg | 206 | M→M | 69 |
| tct→ttt | 269 | S→S | 90 |
| aaa→aag | 576 | | |
| gta→gca | 779 | V→V | 260 |
| tca→tcg | 966 | | |
| gat→gag | 981 | D→D | 327 |
| acg→act | 1137 | | |
| tac→tat | 1149 | | |
| gtg→gta | 1221 | | |
| ctg→ctt | 1410 | | |
| gaa→gag | 1458 | | |
| acc→act | 1602 | | |
| cta→ctg | 1656 | | |
| aac→aat | 1677 | | |
| ttg→ctg | 1692 | | |

Hereinafter, all procedures were performed using a truncated version of the SLO gene (ΔN32 gene) having the nucleotide sequence of SEQ ID NO: 4, which encodes ΔN32 fragment (542 amino acids) having the amino acid sequence of SEQ ID NO: 3 lacking 32 amino acids at the N-terminal of the full-length SLO gene (574 amino acids).

Bax and Bcl-X_{L} constructs were generated in accordance with a conventional method (see [Ko J K, et al., Oncogene 22:2457-65, 2003]), and then ΔN32, Bax and Bcl-X_{L} constructs were cloned into vector pSR α HA (kindly provided by Professor Cheol O, Joe of KAIST), respectively, in accordance with the method of <4-1> of Example 4 below.

### <1-2> Assay of Cell Death Using GFP Co-Transfection

In order to examine whether the overexpressed SLO can induce cell death in 293T cells (ATCC No. CRL-1573), the cells were transfected with the expression vectors prepared in <1-1> above, as follows.

293T cells were cultured in DMEM (Dulbeco's modified Eagle's medium, Invitrogen) supplemented with 10% FBS (fetal bovine serum) at 37° C. in a humidified atmosphere at 5% CO₂. The day before transfection, 5×10⁵/well of 293T cells were plated in a six-well culture plate (Nalgen Nunc International, USA). The next day, 500 ng of each expression construct of HA-ΔN32, Bcl-X_{L} and Bax, and vector pSR α HA as a mock vector were co-transfected with the vector pEGFPC (Clontech, USA) to 293T cells using LipofectAMINE plus® reagent according to the manufacturer's protocol (Invitrogen), respectively. After 24 hours, cells were harvested, washed once with PBS, and the intensity of green fluorescence was monitored by fluorescence-activated cell sorting (FACS) to assay cell death (see [Yang W S, et al., J. Cell Biochem. 94:1234-47, 2005]).

The FACS profile of control transfection experiments (pEGFPC+pSR α HA) showed a peak at a position >4×10² on the X-axis. GFP-expressing cells located within this region were defined as GFp^{high} cells, and 35% of total cells were GFP^{high}. The percentage of dying/dead cells was also determined by monitoring the DNA contents of treated cells by FACS.

After 16 hours of transfection, morphological changes of the 293T cells were monitored under the microscope. As shown in FIG. 1A, the 293T cells transiently transfected with ΔN32 gene became small in size and detached from the culture plate. These results suggest that the expression of ΔN32 can induce cell death. Transfection with proapoptotic Bax produced a similar morphology, but anti-apoptotic Bcl-X_{L} and control mock vector did not change the cell morphology.

293T cells were co-transfected with a GFP reporter and each expression vector and fluorescence intensity was measured by FACS to estimate the viability of the transfected cells. As shown in FIG. 1B, the expression of death-inducing Bax or ΔN32 gradually lowered the fluorescence intensity (that is the percentage of GFP^{high} cells) in transfected cells as the death of transfected cells was increased, whereas the cells transfected with the mock vector and Bcl-X_{L} expression vector maintained similar levels of fluorescence intensity independent of the amount of expression plasmid added. Therefore, the SLO expressed in 293T cells was found to be cytotoxic as similar as a bacterial SLO toxin despite the differences between the bacterial and mammalian expression systems.

### <1-3> Immunoblot

The cells of <1-2> transfected with expression vectors of ΔN32, Bcl-X_{L} and Bax, respectively, were harvested and centrifuged at 500×g for 10 min at 4° C. The cell pellets obtained were washed once with 1 ml of PBS and lysed with 100 μl of 2× sample buffer (20 mM Tris (pH 8.0), 2 mM EDTA, 2% SDS, 20 mM DTT, 1 mM Na₃VO₄, and 20% glycerol). Lysates were sonicated impulse mode, boiled for 5 min at 100° C., centrifuged at 10,000×g for 10 min at 4° C., and the supernatants obtained were used as whole cell lysates. Protein quantification assays were done using MicroBCA reagent (Pierce, USA) according to the manufacturer's protocol. Specifically, 50 μg of total cellular proteins were separated by SDS-PAGE and the separated proteins were then transferred to a nitrocellulose membrane which was then subjected to standard Western blot using mouse anti-HA (Lab Vision, Fremont), mouse anti-Bcl-X_{L} (Santa Cruz Biotechnology, USA), mouse anti-Bax (Santa Cruz Biotechnology, USA) and horseradish peroxidase-conjugated secondary antibodies (Santa Cruz Biotechnology, USA) to monitor the expression levels of all expression plasmids. All blotting membranes were stained with AmidoBlack (Sigma, USA) to ensure that equal amounts of proteins had been loaded into each well. The results are shown in FIG. 1C.

As can be seen in FIG. 1C, the expression level of the SLO protein in 293T cells was increased in proportion to the concentration of the transfected SLO gene.

### EXAMPLE 2

### Assay of Plasma Membrane-Disrupting Effect of SLO

### <2-1> Monitoring Cell Permeability

SLO toxin secreted by bacteria creates large pores in the target cell plasma membrane, and thus, large molecules that normally cannot pass through the membrane are able to freely pass. The present inventors wanted to determine whether the observed cytotoxicity in ΔN32-transfected cells was the result of the increase of the membrane permeability of membrane pores generated by the expressed ΔN32. Thus, the increase of the membrane permeability of plasma membrane was monitored by detecting the level of LDH (lactate dehydrogenase) release from the cytosol of transfected cells into the culture medium. LDH has a molecular weight of ˜125 kDa, and cannot freely transverse the plasma membrane, thus, the LDH release assay has been widely used to measure the level of cell lysis caused by membrane attack by perforin, complement system, or pore-forming toxins.

Cells transfected with each expression vector as described in <1-2> of Example 1 were harvested, and the amount of LDH released from the cells was measured using CytoTox 96 non-radioactive cytotoxicity assay kits (Promega) according to the manufacturer's protocol.

As can be seen in FIG. 2A, the expression of control mock vector caused a release of <10% of LDH, and Bax expression induced the release of 23% of LDH after 24 hours of transfection. In contrast, the level of LDH release due to ΔN32 expression was significantly high (˜60%) indicating that the plasma membrane is readily permeabilized by pore-forming activity of ΔN32. Moreover, the release of LDH in ΔN32-transfected cells was more rapid than that of Bax-transfected cells. Bax induces cell death via apoptotic pathways, which usually retain an intact plasma membrane until the late phase. In contrast, the membrane integrity of ΔN32-transfected cells was rapidly disrupted, which implies that a cell death pathway other than apoptosis is activated during ΔN32-induced cell death.

Further, to assay the change of the membrane permeability in transfected 293T cells by measuring the amount of propidium iodide (PI) uptake, cells transfected as described in <1-2> of Example 1 were harvested and centrifuged at 500×g for 10 min at 4° C. The cell pellets obtained were washed once with 1 ml of PBS and resuspended in 300 μl of PBS. Cells were stained with 0.2 μg/ml of PI and the percentage of PI-permeable cells were measured by FACS.

As can be seen in FIG. 2B, the rapid disruption of plasma membrane integrity was also monitored by the PI uptake experiment. ΔN32-expressing cells were readily stained by exogenous PI dye, which cannot penetrate intact cell membranes. Bax-expressing cells were also stained by PI, but the level of staining was significantly lower than that of ΔN32-expressing cells.

### <2-2> Electron Microscopy

To differentiate Bax-induced cell death and ΔN32-induced cell death more clearly, the ultrastructures of transfected cells were monitored under an electron microscope.

Specifically, Cells transfected as described in <1-2> of Example 1 were washed in PBS, fixed in 2.5% glutaraldehyde at 4° C. for 30 min and postfixed in 1% OSO₄ at room temperature for 20 min. The cells were then dehydrated using increasing concentrations of ethanol (Sigma, USA) (30%, 50%, 70% plus 1% uranylacetate, 80%, 95%, and 100%), embedded in mixtures of Epon/ethanol (Sigma, USA) (1:3 for 15 min, 1:2 for 30 min, 3:1 for 30 min, and pure Epon for 2×30 min), and polymerized at 60° C. for 2 days. The solidified blocks were cut into slices of 50 to 60 nm thickness, which were then contrasted with 5% uranylacetate and Reynold's solution (80 mM Pb(NO₃)₂, 120 mM sodium citrate, 160 mM NaOH) and then viewed under the electron microscope (×100,000).

As can be seen in FIG. 2C, after 16 hours of transfection, Bax-expressing cells had a shrunken appearance, shed fragmented cell debris in a budding-like manner, and maintained a dense cytoplasm. However, ΔN32-expressing cells showed many vacuoles and a less dense cytoplasm, which implies that ΔN32 induces nonapoptotic cell death, necrosis, by creating pores in the plasma membrane.

### EXAMPLE 3

### Biochemical Assay of SLO-Induced Cell Death

### <3-1> Caspase Activity Assay

Caspases are proteolytic enzymes that are activated during various forms of cell death. To test the possibility that caspases are involved in ΔN32-induced cell death, the present inventors measured cellular caspase activities using the caspase-3-specific peptide substrate DEVD-pNA (Calbiochem, USA).

Specifically, 293T cells were transfected with 200, 400, 1,000 ng of each expression construct of Bax, GFP-caspase-8 and HA-ΔN32, and mock vector, and incubated for 24 hours. The GFP-caspase-8 construct was kindly provided by Dr. Miyashita (National Research Institute for Child Health and Development, Japan). Pan-caspase inhibitor, Boc-D was added to the culture at a final concentration of 100 μM. The cultured monolayer cells were harvested and then centrifuged at 450×g for 10 min at 4° C. After removing the supernatant, cell pellets were resuspended in 100 μl of cell lysis buffer (50 mM HEPES (pH 7.5), 1 mM DTT, 0.1 mM DETA, and 0.1% CHAPS) and lysed by repeated freezing at −70° C. and thawing on ice three times. Lysates were then cleared by centrifugation at 15,000×g for 20 min at 4° C., and the resulting supernatants were used as cell extracts. The Caspase assay buffer (100 mM HEPES (pH 7.5), 10% sucrose, 0.1% CHAPS, 10 mM DTT, and 200 μM DEVD-pNA) was added to 100 μg of each cell extract, and the mixtures were then incubated at 37° C. for 4 hours and the yellowish color caused by the release of pNA was quantified using an ELISA reader at 405 nm.

As can be seen in FIG. 3A, the classical proapoptotic proteins Bax and caspase-8, activated cellular caspase-3, whereas treatment with the pan-caspase inhibitor Boc-D, prevented this activation. In contrast, ΔN32 did not activate cellular caspase-3, indicating that ΔN32-induced cell death does not involve caspase-3 activation.

### <3-2> DNA Fragmentation Assay

To analyze DNA fragmentation, cells were transfected as described in <3-1>, incubated for 24 hours, harvested, and centrifuged at 450×g for 10 min at 4° C. The obtained cell pellets were washed once with PBS, and genomic DNA was extracted with DNAzol solution (Molecular Research Center, USA) according to the manufacturer's protocol. The extracted DNA was precipitated with an equal volume of isopropanol, treated with 0.1 mg/ml of RNase A at 37° C., subjected to electrophoresis on 2% agarose gel, and detected by ethidium bromide staining.

Further, protein extracts were prepared from the same harvested cells, and then the cleavage of poly (ADP-ribose) polymerase (PARP) was monitored by Western blot analysis with anti-poly (ADP-ribose) polymerase antibody (Upstate, USA). Pan-caspase inhibitor Boc-D (Calbiochem, USA) was added to each culture of HA-ΔN32, Bax and caspase-8 transfected cell at a final concentration of 100 μM.

As can be seen in FIG. 3B, a representative substrate of caspase-3, PARP was cleaved into 80 kDa fragment as a result of caspase-3 activation when Bax and caspase-8 was expressed, whereas PARP remained intact size (116 kDa) when ΔN32 was expressed. Interestingly, nuclear DNA of ΔN32-expressing cells fragmented into multiples of 200 bp nucleosome units, as demonstrated by agarose gel DNA laddering pattern. Bax and caspase-8-induced internucleosomal DNA cleavage was inhibited by treatment with pan-caspase inhibitor Boc-D, but the ΔN32-induced internucleosomal DNA cleavage was not inhibited even after Boc-D was added to the culture indicating that DNases apart from caspase network, such as serum DNase1, are responsible for this cleavage (see [Napirei M, et al., Arthritis Rheum. 50:1873-83, 2004]).

This result coincided with previous demonstration that treatment of SLO toxin to MDCK cells results in the DNA laddering without cellular cystein protease activation (see [Dong Z, et al., Am. J. Pathol. 151:1205-13, 1997]), which further support the view that ΔN32 synthesized within transfected cells behaved similar to wild type toxin secreted by bacteria.

### <3-3> DNA Quantitative Assay

As SLO-induced cell death seemed to be independent of caspases, the present inventors tested the hypothesis that SLO-induced cell death should not be blocked by the overexpression of potent anti-apoptotic molecules, which is a mechanism commonly used by neoplastic cells to prevent apoptosis (see [Igney P H and Krammer P H, Nat. Rev. Cancer 2:277-88, 2002]).

The inhibition of cell death by cellular anti-apoptotic protein Bcl-X_{L}, viral anti-apoptotic protein CrmA or peptide pan-caspase inhibitor Boc-D was assayed by measuring the green fluorescence emitted by co-expressed GFP reporter protein by FACS as follows. At this time, cDNA fragments encoding CrmA were amplified by PCR and subcloned into vector pFlag-CMV2 (Sigma, USA) to generate FL-CrmA (see [Yang W S, et al., J. Cell Biochem. 94:1234-47, 2005]).

293T cells were transfected with 500 ng of each Bax, GFP-caspase-8, or HA-ΔN32 expression construct. The expression construct for Bcl-X_{L} or CrmA (500 ng) was co-transfected with each of the cell death-inducing DNA constructs, or pan-caspase inhibitor Boc-D was added to the culture at a final concentration of 100 μM. To analyze DNA content, the cells were harvested and washed once with PBS. The cell pellet was fixed overnight with 70% ethanol, washed once with PBS, resuspended in a staining buffer (10 μg/ml PI and 0.2 mg/ml RNase A in PBS), and the resulting cell suspension was kept at 4° C. in the dark until FACS analysis.

As can be seen in FIG. 3C, Bax-induced cell death was inhibited by the overexpression of Bcl-X_{L} and caspase-8-induced cell death was inhibited by the overexpression of Crm A. However, ΔN32-induced cell death was not affected by the overexpression of either of these anti-apoptotic molecules. Even the pan-caspase inhibitor Boc-D did not affect ΔN32-induced cell death. These data show that the SLO protein expressed within 293T cells kills them via a necrotic pathway.

### EXAMPLE 4

### SLO Deletion Assay

### <4-1> Preparation of Deletion Construct

Several deletion mutants of the SLO were generated in order to identify the region responsible for the observed ΔN32-induced cell death.

The DNA fragments of the SLO corresponding to amino acids 33 to 574, 106 to 574, 116 to 574, 151 to 574, 1 to 569 and 1 to 530 of the SLO protein (Genbank accession number, AB050250) were amplified by PCR using ExTaq Polymerase (Takara Bio), primers (SEQ ID NOs: 5 to 16) and pcDNA3-SLO prepared in <1-1> of Example 1 as a template. The PCR condition was 30 cycles of 5 min at 94° C., 1 min at 58° C. and 1 min at 72° C. after initial denaturation for 10 min at 94° C., and final extension for 1 min at 72° C. The amplified DNA fragments, ΔN32, ΔN105 and ΔC44 were subcloned into EcoRI/XhoI restriction site of vector pcDNA3 (Invitrogen), ΔN115 and ΔN150 fragments were subcloned into EcoRI/EcoRV restriction site of the vector, and ΔC5 fragment was subcloned into EcoRV/XhoI restriction site of the vector to generate pcDNA3-ΔN32, pcDNA3-ΔN105, pcDNA3-ΔN115, pcDNA3-ΔN150, pcDNA3-ΔC5 and pcDNA3-ΔC44, respectively. In order to detect expressed SLO derivatives using anti-HA antibodies (Lab Vision, USA), all the cloned vectors were cleaved with EcoRI and XhoI restriction enzymes (Promega, USA), and the cleaved fragments were subcloned between the EcoRI and SalI sites of vector pSRαHA. The subcloned SLO fragment was analyzed by the dideoxynucleotide sequencing method.

As can be seen in a diagram of the deletion constructs prepared above of FIG. 4A, all deletion constructs were fused to HA tag at the N-terminus to be detected by anti-HA antibodies, and included transmembrane helices 1 and 2 (TMH1 and TMH2), and domain 4 as the cholesterol binding domain of the SLO.

### <4-2> Assay of Cell-Killing Activity of Deletion Constructs

The expression of each deletion construct was monitored by Western blot analysis using anti-HA antibody. Cells grown in six-well culture plates were transfected with 500 ng of each DNA construct, incubated for 24 hours and harvested. After performing Western blot analysis using anti-HA antibody as described in <1-3> of Example 1, the blotting membrane was stripped and reblotted with antibody against ERK ½ (Upstate Biotechnology, USA) to monitor the amount of protein loaded onto the protein gel.

Further, the degree of plasma membrane permeabilization was monitored using an LDH release assay kit as described in <2-1> of Example 2. The cells were stained with PI, and the percentage of PI-permeable cells including sub-genomic DNA contents caused by DNA fragmentation was measured by FACS to monitor the degree of cell death.

As can be seen in FIGS. 4C and 4D, the full-length SLO protein fused with HA-tag permeabilized cell membrane and killed transfected cells. However, the present inventors could not detect the HA-SLO protein by Western blotting with anti-HA antibody, suggesting that the posttranslational modification of eukaryotic cell was processed at the end of the N-terminal region of the SLO.

As can be seen in FIGS. 4C and 4D, deletions up to 115 amino acids at the N-terminus of SLO induced cell death when expressed in 293T cells. However, when 150 amino acids at the N-terminus were removed, the cell-killing activity of the SLO disappeared almost completely. This indicates that amino acids 116 to 150 of N-terminus of the SLO protein include an essential region for the SLO-induced membrane permeabilization. However, in contrast to the N-terminus, deletions of only five amino acids at the C-terminus abolished the cell-killing activity of the SLO. These results are similar to the results from the bacterially expressed SLO, in which deletion of N-terminal 107 amino acids had no effect on cytolytic activity of the SLO whereas only one C-terminal amino acid deletion showed marked decrease in the SLO cytolytic activity (see [Yamamoto I, et al., Biosci. Biotechnol. Biochem. 65:2682-9, 2001]). Thus, this deletion study emphasizes the importance of cholesterol binding in cell-killing activity of the SLO and indicates that the SLO protein expressed in mammalian cells behaves like its bacterial counterpart.

### EXAMPLE 5

### Generation of SLO Recombinant Adenovirus

In order to examine the possibility that the SLO gene having cell-killing activity can be used as an anti-cancer gene therapeutic reagent, a replication-deficient adenovirus that can express the ΔN32 gene was generated. Because ΔN32 is a cytotoxic protein, a Cre-inducible system that is widely used for toxic gene expression was used to circumvent the difficulties of virus packaging in the 293T cell line.

Specifically, the stuffer DNA fragment flanked by two loxp sequences was amplified from the pDNR-CMV plasmid (Clontech) using ExTaq Polymerase (Takara Bio) and a pair of primers (SEQ ID NOs: 17 and 18). The amplified product was cloned into the XbaI site of shuttle vector pCA14 (kindly provided from Professor Chae-Ok, Yun of Yonsei university, Korea) to generate the pCA14-loxP construct. The GFP gene from pEGFPC1 plasmid (Clontech) was amplified using ExTaq Polymerase (Takara bio) and a pair of primers (SEQ ID NOs: 19 and 20). The PCR condition was 30 cycles of 5 min at 94° C., 1 min at 58° C. and 1 min at 72° C. after initial denaturation of 10 min at 94° C., and final extension of 1 min at 72° C.

The amplified DNA fragment was cloned into the EcoRI/SalI site of shuttle vector pCA14-loxP to generate a recombinant GFP expression vector, pCA14-loxP-GFP. cDNA encoding the deletion fragment of the SLO (ΔN32) was excised from pcDNA3-ΔN32 plasmid using EcoRI and XhoI restriction enzymes and cloned into the EcoRI/SalI site of shuttle vector pCA14-loxP to generate an recombinant SLO expression vector pCA14-loxP-SLO.

The pCA14-loxP-GFP and pCA14-loxP-SLO constructs were then linearized by XmnI and PvuI digestion, respectively, and the adenoviral vector vmdl324Bst (obtained from S. B. Verca of University of Fribourg, Switzerland) containing the Ad5 genome lacking E1 and E3 regions was linearized by BstBI digestion. The linearized pCA14-loxP-GFP and pCA14-loxP-SLO were co-transformed into E. Coli BJ5183 (obtained from Professor Chae-Ok, Yun of Yonsei university, Korea) together with the BstBI-digested vmdl324Bst for homologous recombination. To verify the respective homologous recombinants, plasmid DNA, purified from overnight E. Coli culture, was digested with HindIII and the digestion pattern was analyzed. Proper homologous recombinant adenoviral plasmid DNA was digested with PacI and transfected into 293T cells.

Ten days after the transfection into the 293T cells, the cell culture supernatants showing the evident cytopathic effect were harvested and centrifuged to obtain a clear culture supernatant. Aliquots were analyzed by PCR using SLO-specific primers (SEQ ID NOs: 19 to 22) and DNA sequencing analysis to confirm the presence of a recombinant adenovirus encoding loxP-GFP or loxP-ΔN32GFP in the transfected cells. These adenoviruses were further transfected to 293T cells for amplification, purified using standard CsCl gradient methods, and designated to Ad-loxP-GFP and Ad-loxP-SLO. The titer (multiplicity of infection, MOI) used in the present invention hereinafter was determined by the absorbance of the dissociated virus at 260 nm, where 1 absorbance unit is equivalent to 10¹² viral particles per ml. At this time, the particle-to-infectious unit (IU) ratio was 100:1.

To test the functionality of the purified adenovirus, the present inventors infected the adenovirus to the human cervical carcinoma C33A cell line and monitored the green fluorescence emitted by GFP. C33A cells (ATCC HTB-31) were cultured in the wells of a 24-well plate containing DMEM (Invitrogen) supplemented with 10% FBS at 37° C. in a humidified atmosphere at 5% CO₂. The cultured cells were infected with 10 MOI of Ad-loxP-GFP or Ad-loxP-ΔN32 with or without 5 MOI of Cre-expressing adenovirus, AdCreM2 virus (Microbix Biosystems Inc), incubated for 3 days and harvested. AdCreM2 was propagated in 293T cells and purified using standard methods (see [Kim E, et al., Hum. Gene Ther. 14:1415-28, 2003]), and the Cre-inducible expression of GFP was monitored by fluorescence microscopy and FACS.

As can be seen in FIG. 5B, infection with only Ad-loxP-GFP, and not Cre virus (AdCreM2), showed a slight increase in the number of green-colored cells, indicating the presence of leaky expression in the loxP system. However, when C33A cells were co-infected with Ad-loxP-GFP and AdCreM2, GFP overexpression was induced and about >80% of the cell population was positive for the green color. This control experiment confirmed the functionality of the purified adenovirus and of the Cre-loxP system.

Therefore, in order to examine the functionality of the SLO-expressing recombinant adenovirus Ad-loxP-SLO in C33A cells, the cell death induced by Cre-inducible expression of the SLO protein was measured by MTX assay. C33A cells grown in 24-well culture plates were infected with 10 MOI of Ad-loxP-GFP or Ad-loxP-SLO with or without 50 MOI of AdCreM2, incubated for 3 days, and then MTX reagent was added thereto. The ratio of AdCreM2 to Ad-loxP-GFP or Ad-loxP-SLO was 1:2 at all MOIs.

As can be seen in FIG. 5C, co-infection with Ad-loxP-SLO and AdCreM2 caused marked C33A cell death as the virus titer was increased. However, the control Ad-loxP-GFP virus in combination with AdCreM2 showed an intermediate level of cytotoxicity, and Ad-loxP-GFP or Ad-loxP-SLO without AdCreM2 showed little cytotoxicity at about <10 MOI.

As a result, the SLO-expressing recombinant adenovirus of the present invention overexpressing the SLO shows the cell-killing activity under the control of Cre-inducible expression system.

### EXAMPLE 6

### Anti-Tumor Effect of SLO Recombinant Adenovirus In Vitro

In order to examine the anti-tumor effect of the SLO recombinant adenovirus in various cancer cell lines, C33A (ATCC HTB-31), A549 (human lung carcinoma, ATCC CCL-185), MCF-7 (ATCC HTB-22) and PC-3 (ATCC CRL-1435) cell lines were grown in 24-well plates to 30% to 70% confluences, and infected with 10 MOI of Ad-loxP-GFP or Ad-loxP-SLO with or without 5 MOI of AdCreM2. After 6 days, the infected cells were harvested, and the cells were stained with PI and the percentage of dying/dead cells including sub-genomic DNA contents caused by DNA fragmentation was measured by FACS. PBS treated cells were used as a negative control.

As can be seen in FIG. 6, six days after co-infection with Ad-loxp-SLO and AdCreM2, about >90% cell death was observed in C33A and A549 cells, whereas the control PBS group did not show any remarkable cytotoxic effect. However, co-infection with Ad-loxP-GFP and AdCreM2 produced an intermediate level of cytotoxicity in these cells, suggesting that the expression of GFP and/or Cre protein exerts a toxic effect in these cell lines. This result was identical to the reports that expression of GFP or Cre proteins are toxic to cell lines under specific experimental conditions (see [Hanazono Y, et al., Hum. Gene Ther. 8:1313-9, 1997; Liu H S, et al., Biochem. Biophys. Res. Commun. 260:712-7, 1999; Loonstra A, et al., Proc. Natl. Acad. Sci. U.S.A. 98:9209-14, 2001; and Silver D P and Livingston D M, Mol. Cell 8:233-43, 2005]). When MCF-7 and PC-3 cells were used as target cells, about 60% cell death was observed for the same viral dose and time as in C33A and A549 cells. In contrast to C33A and A549 cells, co-infection with the control Ad-loxP-GFP and AdCreM2 viruses showed little cytotoxicity in MCF-7 and PC-3 cells. Thus, differences in the level of cell death in each cell line can be explained by differences in the sensitivity of each cell line to viral infection.

### EXAMPLE 7

### Anti-Tumor Effect of SLO Recombinant Adenovirus In Vivo

The in vivo anti-tumor effect of ΔN32-expressing recombinant adenovirus was evaluated by injecting the SLO recombinant adenovirus of the present invention into tumors established from human cervical cancer xenografts C33A, in nude mice as follows.

Specifically, C33A cells (1×10⁷) were injected into the flanks of 6 to 8-week-old male nude mice (Charles River Japan Inc., Yokohama, Japan) to establish human tumor xenografts. When tumor volumes reached 6 to 7 mm in diameter, mice were randomized into three groups, and 50 μL of PBS solution containing 5×10⁸ PFU (plaque-forming units) of the Ad-loxP-GFP or Ad-loxP-SLO virus was injected directly into the tumors together with 25 μl of PBS solution containing 2.5×10⁷ PFU of the AdCreM2 virus twice/day. There was no appearance of any leakage of viral solution into surrounding tissue. Control tumors were injected with PBS only. Tumor growth was measured twice or thrice times weekly until the end of the study by measuring the length and width of each tumor using calipers. Tumor volumes were estimated using the following formula:

Tumor Volume=0.523 LW²

wherein, L is the longitudinal length of a tumor, and W is the lateral length of a tumor.

As can be seen in FIG. 7, control tumors, which were treated with PBS, increased to an average size of 2412±792 mm³ at 27 days after the virus injection. Whereas, tumor growth was significantly inhibited in mice coinjected with the replication-incompetent Ad-loxP-SLO/AdCreM2, and the average tumor size was 803±328 mm³ at 27 days after the virus injection. Control tumors, which received Ad-loxP-GFP/AdCreM2, showed an intermediate level of growth inhibition, which is consistent with the in vitro data. Throughout the course of this experiment, no systemic toxicity, such as diarrhea, loss of weight, or cachexia was observed.

While the invention has been described with respect to the above specific embodiments, it should be recognized that various modifications and changes may be made and also fall within the scope of the invention as defined by the claims that follow.

1. A recombinant adenovirus expressing a streptolysin O (SLO) protein comprising a SLO gene; a promoter operably linked to the SLO gene; a polyadenylation signal sequence; and an adenovirus genome lacking E1 gene.

2. The recombinant adenovirus of claim 1, wherein the SLO gene is a polynucleotide selected from the group consisting of the polynucleotides encoding 33^{th} to 574^{th}, 106^{th} to 574^{th} and 116^{th} to 574^{th} amino acids of the SLO protein (Genbank accession No. AB0505250), respectively.

3. (canceled)

4. The recombinant adenovirus of claim 2, wherein the polynucleotide encodes the amino acid sequence of SEQ ID NO: 3.

5. The recombinant adenovirus of claim 2, wherein the polynucleotide has a nucleotide sequence of SEQ ID NO: 4.

6. The recombinant adenovirus of claim 1, which is prepared by an in vivo recombination process comprising co-transfecting a mammalian cell with a recombinant vector comprising the SLO gene, the promoter and the polyadenylation signal sequence, and a viral vector comprising an adenovirus genome lacking E1 gene.

7. An anti-cancer composition comprising the recombinant adenovirus claim 1 as an active ingredient.


SEQUENCE LISTING (22 sequences)

SEQ ID NO: 1 - DNA, 38 bp, Artificial Sequence
  forward primer slo 1 up
gatcccgaattcatgaaggacatgtctaataaaaaaac

SEQ ID NO: 2 - DNA, 37 bp, Artificial Sequence
  reverse primer slo 574 dn
gatcccctcgagctacttataagtaatcgaaccatat

SEQ ID NO: 3 - protein, 542 aa, Artificial Sequence
  SLO protein having 32 amino acid deletion
NAESNKQNTASTETTTTSEQPKPESSELTIEKAGQKMDDMLNSNDMIKLAPKEMPLESAEKEEKKSEDKKKSEEDHTEEINDKIYSLNYNELEVLAKNGETIENFVPKEGVKKADKFIVIERKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFKDLQRKGVSNEAPPLFVSNVAYGRTVFVKLETSSKSNDVEAAFSAALKGTDVKTNGKYSDILENSSFTAVVLGGDAAEHNKVVTKDFDVIRNVIKDNATFSRKNPAYPISYTSVFLKNNKIAGVNNRTEYVETTSTEYTSGKINLSHQGAYVAQYEILWDEINYDDKGKEVITKRRWDNNWYSKTSPFSTVIPLGANSRNIRIMARECTGLAWEWWRKVIDERDVKLSKEINVNISGSTLSPYGSITYK

SEQ ID NO: 4 - DNA, 1629 bp, Artificial Sequence
  SLO gene fragment encoding SLO protein having 32 amino acid deletion
aatgctgaatcgaacaaacaaaacactgctagtacagaaaccacaacgacaagtgagcaaccaaaaccagaaagtagtgagctaactatcgaaaaagcaggtcagaaaatggatgatatgcttaactctaacgatatgattaagcttgctcccaaagaaatgccactagaatctgcagaaaaagaagaaaaaaagtcagaagacaaaaaaaagagcgaagaagatcacactgaagaaatcaatgacaagatttattcactaaattataatgagcttgaagtacttgctaaaaatggtgaaaccattgaaaattttgttcctaaagaaggcgttaagaaagctgataaatttattgtcattgaaagaaagaaaaaaaatatcaacactacaccagtcgatatttccattattgactctgtcactgataggacctatccagcagcccttcagctggctaataaaggttttaccgaaaacaaaccagacgcggtagtcaccaagcgaaacccacaaaaaatccatattgatttaccaggtatgggagacaaagcaacggttgaggtcaatgaccctacctatgccaatgtttcaacagctattgataatcttgttaaccaatggcatgataattattctggtggtaatacgcttcctgccagaacacaatatactgaatcaatggtatattctaagtcacagattgaagcagctctaaatgttaatagcaaaatcttagatggtactttaggcattgatttcaagtcgatttcaaaaggtgaaaagaaggtgatgattgcagcatacaagcaaattttttacaccgtatcagcaaaccttcctaataatcctgcggatgtgtttgataaatcagtgacctttaaagatttgcaacgaaaaggtgtcagcaatgaagctccgccactctttgtgagtaacgtagcctatggtcgaactgtttttgtcaaactagaaacaagttctaaaagtaatgatgttgaagcggcctttagtgcagctctaaaaggaacagatgttaaaacgaatggaaaatactctgatatcttagaaaatagctcatttacagctgtcgttttaggaggagatgctgcagagcacaataaggtggtcacaaaagactttgatgttattagaaacgttatcaaagacaatgctaccttcagtagaaaaaacccagcttatcctatttcatacaccagtgttttccttaaaaataataaaattgcgggtgtcaataacagaactgaatacgttgaaacaacatctaccgagtacactagtggaaaaattaacctgtctcatcaaggcgcgtatgttgctcaatatgaaatcctttgggatgaaatcaattatgatgacaaaggaaaagaagtgattacaaaacgacgttgggacaacaactggtatagtaagacatcaccatttagcacagttatcccactaggagctaattcacgaaatatccgtatcatggctagagagtgcaccggcttagcttgggaatggtggcgaaaagtgatcgacgaaagagatgtgaaactatctaaagaaatcaatgtcaacatctcaggatcaaccttgagcccatatggttcgattacttataagtag

SEQ ID NO: 5 - DNA, 43 bp, Artificial Sequence
  slo33up forward primer for pcDNA3-N32
gatcccgaattcatgaatgctgaatcgaacaaacaaaacactg

SEQ ID NO: 6 - DNA, 37 bp, Artificial Sequence; identical to SEQ ID NO: 2
  slo 574 dn reverse primer for pcDNA3- N32

SEQ ID NO: 7 - DNA, 39 bp, Artificial Sequence
  slo 106 up forward primer for pcDNA3-N105
gatcccgaattcatggaagatcacactgaagaaatcaat

SEQ ID NO: 8 - DNA, 37 bp, Artificial Sequence; identical to SEQ ID NO: 2
  slo 574 dn reverse primer for pcDNA3-N105

SEQ ID NO: 9 - DNA, 42 bp, Artificial Sequence
  slo116up forward primer for pcDNA3-N115
gatcccgaattcatgatttattcactaaattataatgagctt

SEQ ID NO: 10 - DNA, 22 bp, Artificial Sequence
  sloseqdn-1 reverse primer for pcDNA3-N115
actgaaggtagcattgtctttg

SEQ ID NO: 11 - DNA, 42 bp, Artificial Sequence
  slo151up forward primer for pcDNA3-N150
gatcccgaattcatgattgaaagaaagaaaaaaaatatcaac

SEQ ID NO: 12 - DNA, 37 bp, Artificial Sequence; identical to SEQ ID NO: 2
  slo 574 dn reverse primer for pcDNA3-N150

SEQ ID NO: 13 - DNA, 42 bp, Artificial Sequence
  slo 115 up forward primer for pcDNA3-C5
gatcccgaattcatgaagatttattcactaaattataatgag

SEQ ID NO: 14 - DNA, 37 bp, Artificial Sequence
  slo569dn reverse primer for pcDNA3-C5
gatcccctcgagctaaccatatgggctcaaggttgat

SEQ ID NO: 15 - DNA, 38 bp, Artificial Sequence; identical to SEQ ID NO: 1
  slo 1 up forward primer for pcDNA3-C44

SEQ ID NO: 16 - DNA, 38 bp, Artificial Sequence
  slo531dn reverse primer for pcDNA3-C44
gatcccctcgagctaagccatgatacggatatttcgtg

SEQ ID NO: 17 - DNA, 32 bp, Artificial Sequence
  forward primer loxup
gatccctctagagctagcgtcagtgagcgagg

SEQ ID NO: 18 - DNA, 32 bp, Artificial Sequence
  reverse primer loxdn
gatccctctagagtcgcggccgcttaacttcg

SEQ ID NO: 19 - DNA, 31 bp, Artificial Sequence
  forward primer GFPRIup
gatcccgaattcatggtgagcaagggcgagg

SEQ ID NO: 20 - DNA, 39 bp, Artificial Sequence
  reverse primer GFPSaldn
gatagacccgtcgactcaacttgtacagctcgtccatgc

SEQ ID NO: 21 - DNA, 43 bp, Artificial Sequence; identical to SEQ ID NO: 5
  forward primer slo33up

SEQ ID NO: 22 - DNA, 37 bp, Artificial Sequence; identical to SEQ ID NO: 2
  reverse primer slo 574 dn